(19) United States
(12) Patent Application Publication
(10) Pub. No.: US 2022/0072276 A1
(43) Pub. Date: Mar. 10, 2022

(54) CATHETER ASSEMBLY

(71) Applicant: TELEFLEX LIFE SCIENCES UNLIMITED COMPANY, Athlone (IE)

(72) Inventors: Aleksejus FOMINAS, Athlone (IE); Cathal TIGHE, Athlone (IE); Morgan TIERNEY, Tullamore (IE)

(21) Appl. No.: 17/525,946

(22) Filed: Nov. 14, 2021

Related U.S. Application Data
(63) Continuation of application No. PCT/IB2020/054639, filed on May 15, 2020.
(60) Provisional application No. 62/848,333, filed on May 15, 2019.

(51) Int. Cl. A61M 25/01; A61M 25/00

(52) U.S. Cl. CPC A61M 25/0119; A61M 25/0102; A61M 2025/0081; A61M 25/0074; A61M 25/0111

(57) ABSTRACT

A catheter assembly may include a catheter member having a proximal portion and an outer surface; an introducer having a lumen configured to receive the proximal portion of the catheter member; and a sleeve having a compressed portion housed in the lumen of the introducer, the sleeve being configured to evert over the outer surface as the catheter member is inserted through the lumen of the introducer. The proximal portion of the catheter member may be configured to push a distal portion of the sleeve proximally, and the compressed portion of the sleeve may form a soft proximal tip of the catheter assembly as the catheter member exits a proximal opening of the introducer.

### PRIORITY

The present application is a continuation of International Application No. PCT/IB2020/054639, filed on May 15, 2020, which claims the benefit of priority to U.S. Provisional Patent Application No. 62/848,333, filed on May 15, 2019 and now expired, the disclosures of which are expressly incorporated herein in their entirety.

### TECHNICAL FIELD

The present disclosure relates generally to catheters, and more particularly to two-part catheter assemblies configured to reduce friction of a catheter member when introduced into a bodily lumen.

### BACKGROUND

Urinary catheters are often used to void urine from a patient's bladder by being inserted through a urethra and into the bladder. Friction between the catheter and the urethra can cause discomfort during insertion and/or removal. The present invention solves one or more problems involving the catheters of the prior art.

### SUMMARY

One aspect of the present invention is directed to a catheter assembly including a catheter member, an introducer, and a sleeve. The catheter member may have a proximal portion and an outer surface. The introducer may have a lumen configured to receive the proximal portion of the catheter member, and the sleeve may have a compressed portion housed in the lumen of the introducer, the sleeve being configured to evert over the outer surface as the catheter member is inserted through the lumen of the introducer.

In some embodiments, the proximal portion of the catheter member is configured to push a distal portion of the sleeve proximally, and the compressed portion of the sleeve forms a soft proximal tip of the catheter assembly as the catheter member exits a proximal opening of the introducer. In some embodiments, the sleeve is not secured to the catheter member. In some embodiments, the introducer includes a cap member having an enlarged proximal surface. In some embodiments, the cap member has a retaining member that covers the sleeve when contained in the introducer and deflects as the catheter member is advanced proximally from the introducer. In some embodiments, the retaining member includes a plurality of flaps. In some embodiments, the retaining member forms a projection extending proximally from the enlarged proximal surface. In some embodiments, the introducer includes a handle member having a surface configured to be gripped by a user. In some embodiments, the surface of the handle member includes a concave surface and/or one or more ridges. In some embodiments, the proximal portion of the sleeve is secured to the introducer. In some embodiments, the cap member and the handle member are in a mating configuration. In some embodiments, the distal portion of the sleeve is releasably engaged by an internal shoulder of the introducer. In some embodiments, the handle member includes a plurality of elongated guide members extending from a distal portion. In some embodiments, the proximal portion of the catheter member is open, and the catheter member has a lumen therethrough. In some embodiments, the catheter member includes a funnel member on a distal portion. In some embodiments, the catheter assembly is non-lubricated.

Another aspect of the present invention is directed to a method of inserting the catheter assembly into a bodily lumen. The method may include inserting the catheter member into the lumen of the introducer containing the compressed portion of the sleeve, and everting the sleeve over the catheter member as the catheter member is inserted into the bodily lumen.

In some embodiments, the method may further include pushing a distal portion of the sleeve proximally, and forming a soft proximal tip of the catheter assembly with the compressed portion of the sleeve as the catheter member is inserted into the bodily lumen. In some embodiments, the method may further include inserting a dome member of the introducer into the bodily lumen. In some embodiments, the method may further include retaining the sleeve in the introducer with a retaining member, and deflecting the retaining member as the sleeve is advanced proximally from the introducer.

### BRIEF DESCRIPTION OF DRAWINGS

FIG. 1 illustrates a side view of a catheter assembly according to a first embodiment of the present invention.

FIG. 2 illustrates an exploded view of the catheter assembly of FIG. 1.

FIG. 3 illustrates a perspective view of an introducer of the catheter assembly of FIGS. 1-2.

FIG. 4 illustrates a side view of a cap member of the catheter assembly of FIGS. 1-3.

FIG. 5 illustrates a cross-sectional view of the cap member of the catheter assembly of FIG. 4.

FIG. 6 illustrates a cross-sectional view of a sleeve of the catheter assembly of FIGS. 1-5.

FIG. 7 illustrates a side view of a handle member of the catheter assembly of FIGS. 1-6.

FIG. 8 illustrates a cross-sectional view of the introducer of the catheter assembly of FIGS. 1-7.

FIG. 9 illustrates a cross-sectional view of the catheter assembly of FIGS. 1-8.

FIG. 10 illustrates a side view of a catheter member of the catheter assembly of FIGS. 1-9.

FIGS. 11A-D illustrate a method of using the catheter assembly of FIGS. 1-10.

FIGS. 12A-C illustrate views of an introducer of a catheter assembly according to a second embodiment of the present invention.

### DETAILED DESCRIPTION

The present invention is generally directed to catheter assemblies and methods of use including a compact unit or introducer housing a thin pliable membrane or sleeve compressed in a bunched, bellowed, and/or folded configuration. The present invention incorporates a friction free concept into a compact introducer that may be used with any device (e.g., any type of elongated member including open or closed tip tubular members) without being integrated into the device. The present invention also allows for a two-piece concept for intermittent catheters, including a disposable sleeve and a reusable pusher and/or catheter member. The catheter member may be inserted into a bodily lumen (e.g., a urethra) through the disposable sleeve minimizing the friction during insertion, as the introducer translates relatively along an outer surface of the catheter member. The sleeve and the catheter member may be removed together or separately. For example, the catheter member may be first removed through the sleeve, and then the sleeve may be removed reducing any discomfort caused during removal. In some embodiments, the introducer may additionally or alternatively include clamping members that clamp the catheter member for a one step removal. The catheter assembly may be non-lubricated to prevent spilling and staining of clothing of the user or administer. The present invention may be used in any number of applications, including urology applications (e.g., intermittent catheters, Foley catheters, endo devices, delivery systems), vascular applications (e.g., sheaths), and other surgical applications. The terms “proximal” and “distal” as used herein are defined by the relative proximity to the person, animal, object, or other recipient into which the catheter assembly is being inserted. The catheter assembly may be self-administered such that the terms “proximal” and “distal” may refer to the proximity to the administer and the recipient.

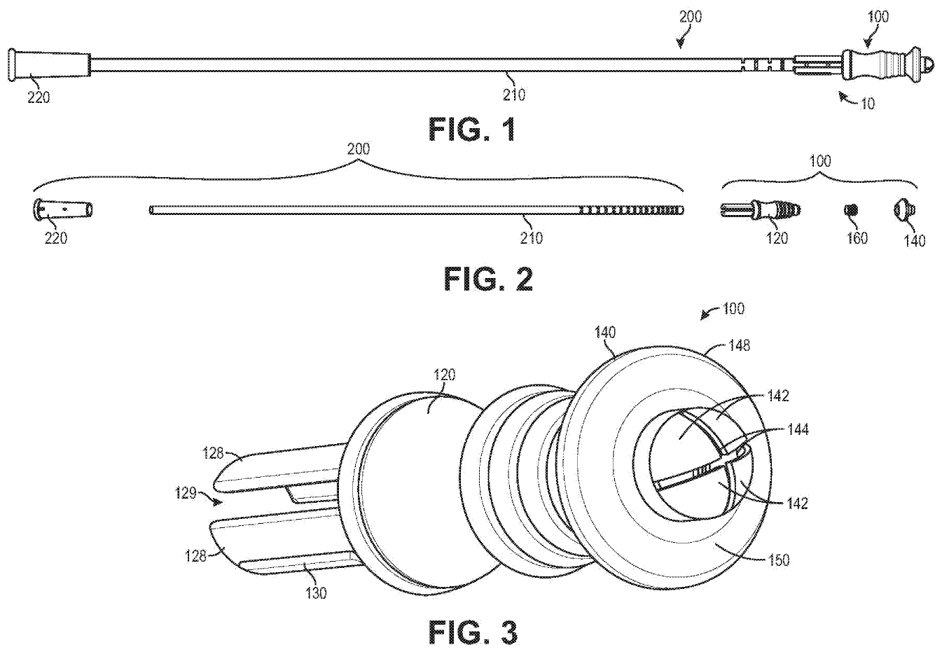

FIG. 1 illustrates a side view of a catheter assembly 10 according to embodiments of the present invention, and FIG. 2 illustrates an exploded view of the catheter assembly 10. As illustrated, the catheter assembly 10 may include an introducer 100 and a catheter member 200. The catheter member 200 may be configured to be inserted into a lumen of the introducer 100 prior to insertion into a bodily lumen. As further illustrated in FIG. 2, the introducer 100 may include a handle member 120, a cap member 140, and a sleeve 160. The cap member 140 may be on a proximal end portion of the handle member 120, and the sleeve 160 may be housed by the handle member 120 and/or the cap member 140. The catheter member 200 may include a tubular member 210 extending proximally from a handle member 220. The handle member 120, the cap member 140, and/or the sleeve 160 may collectively define the lumen of the introducer 100 that receives the tubular member 210 as the tubular member 210 is introduced into the bodily lumen.

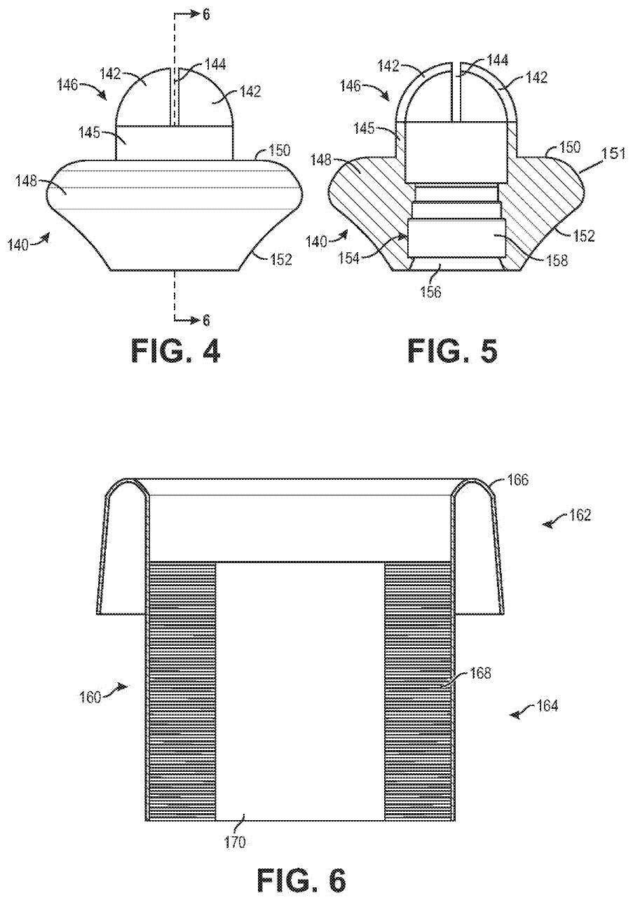

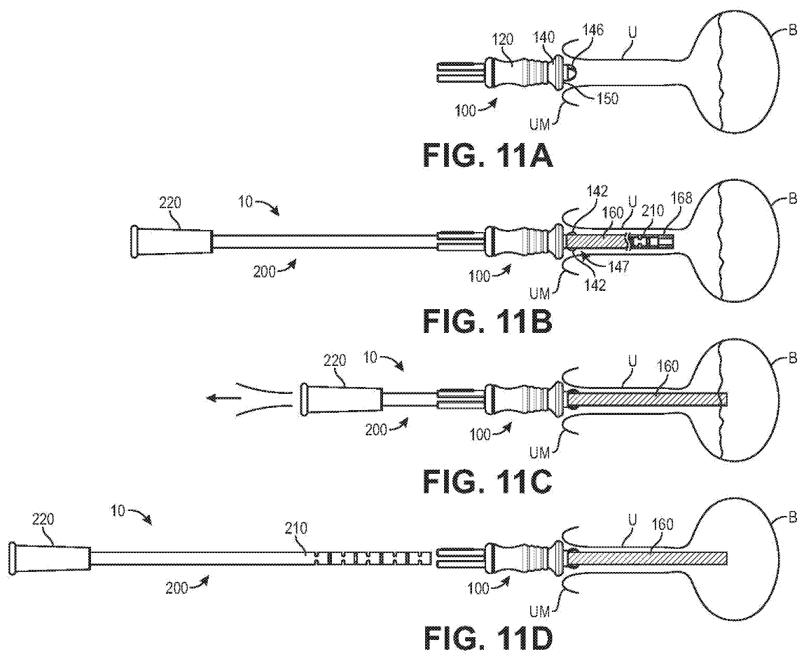

FIG. 3 illustrates an isometric view of the introducer 100, and FIGS. 4-9 illustrate one or more components of the introducer 100. As illustrated in FIGS. 3-5, the cap member 140 may have a retaining member configured to cover and/or retain the sleeve 160 in the introducer 100 during storage. The retaining member may open when a sufficient proximal pushing force is applied by the catheter member 200. The retaining member may include a plurality of flaps 142 separated by preformed radial slots 144 and substantially close a proximal opening 147 (as illustrated in FIG. 11B). The flaps 142 may be resilient to sufficiently cover and/or retain the sleeve 160 within the introducer 100 and/or prevent pathogens from entering the introducer 100 through the proximal opening 147. The flaps 142 may separate or resiliently deflect when pressed proximally as the catheter member 200 exits proximally through the proximal opening 147. The slots 144 may extend distally to a solid tubular wall segment 145. The cap member 140 may form a projection 146 in the form of a narrow portion extending proximally from the cap member 140. The projection 146 may be inserted into an opening of the bodily lumen to align and initially retain the introducer 100 with respect to the bodily lumen. The projection 146 may be a hollow dome structure formed by the flaps 142 and/or the solid tubular wall segment 145. The cap member 140 may further include a flange 148 distal of the projection 146. The flange 148 may have an enlarged, substantially flat proximal surface 150 configured to engage tissue around an opening of the bodily lumen to prevent further insertion of the introducer into the bodily lumen. The flat proximal surface 150 may have angled and/or convex side surfaces 151 to conform to anatomy. The flange 148 may also have an angled and/or concave distal surface 152 to prevent user contact with the projection 146 and resultant introduction of pathogens into the bodily lumen.

As further illustrated in FIG. 5, the cap member 140 may have a lumen 154 extended therethrough for receiving the handle member 120, the sleeve 160, and the catheter member 200. The lumen 154 may include a distal opening 156 having tapered walls that facilitates insertion and retention of a proximal end portion of the handle member 120. The lumen 154 may also have a widened region 158 configured to receive the proximal end portion of the handle member 120. The widened region 158 may be wider than the distal opening 156 to provide an interference and/or snap-fit to retain the handle member 120 and prevent detachment.

FIG. 6 illustrates a cross-sectional view of the sleeve 160. The sleeve 160 may have a proximal end portion 162 and a distal end portion 164, and the entirety of the sleeve 160 may be housed in the introducer 100 prior to insertion of the catheter member 200. The proximal end portion 162 may be secured to the introducer 100, and the distal end portion 164 may be configured to be pushed by the catheter member 200 proximally from the introducer 100 through the bodily lumen. Thus, the proximal end portion 162 may have a folded portion 166 secured to the introducer 100 by being sandwiched between the handle member 120 and the cap member 140. The distal end portion 164 may have a compressed portion 168 releasably housed in the introducer 100 prior to insertion of the catheter member 200. The sleeve 160 may be housed such that the proximal end portion of the catheter member 200 engages and pushes the distal end portion 164 of the sleeve 160. The compressed portion 168 may unfold and evert over the catheter member 200 as the catheter member 200 exits the proximal opening 147 and passes through the bodily lumen. The compressed portion 168 may form a soft proximal tip on the proximal end portion of the catheter member 200 as the catheter member 200 is advanced through the bodily lumen. The compressed portion 168 may be compressed and/or folded in a uniform configuration maintaining a lumen 170 therethrough, thus allowing passage of fluids without the sleeve 160 being completely unfolded. This may allow the compressed portion 168 to form the soft proximal tip on the catheter member 200 through the entire length of the bodily lumen.

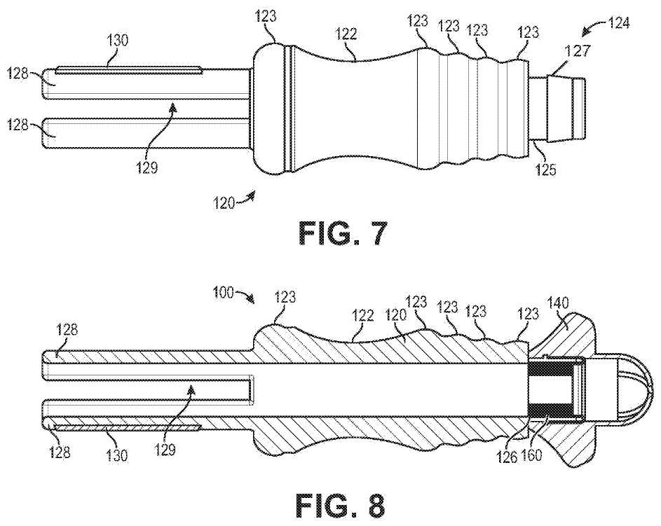

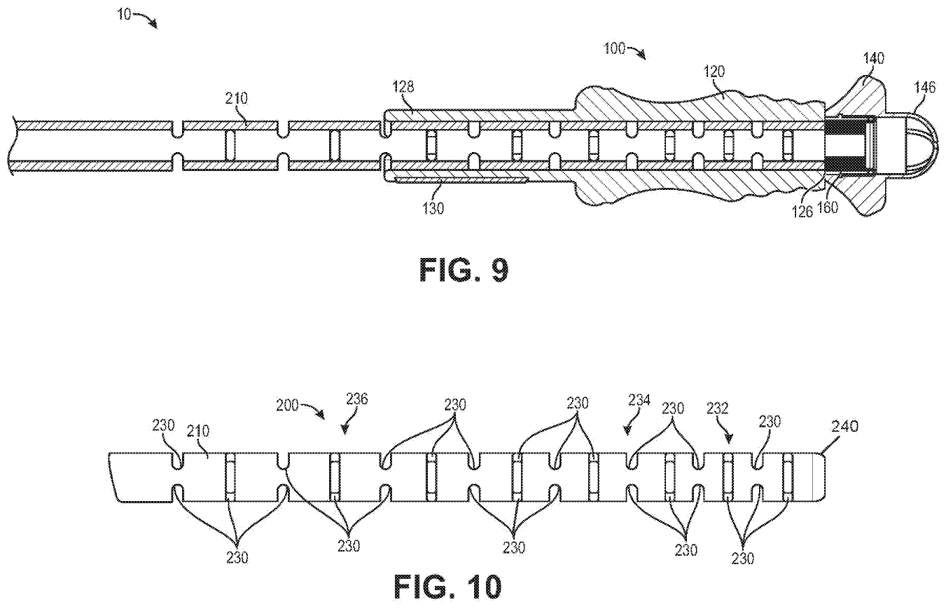

FIG. 7 illustrates a side view of the handle member 120, FIG. 8 illustrates a cross-sectional view of the introducer 100, and FIG. 9 illustrates a cross-sectional view of the catheter assembly 10. The handle member 120 may have a concave and/or textured surface configured to be gripped by the user, for example, formed by a concave surface 122 and/or one or more circumferential ridges 123. The handle member 120 may further include a proximal end portion 124 received in the lumen 154 of the cap member 140 in a mating configuration. The proximal end portion 124 may include a male member 125 having a barb 127 configured to be inserted through the distal opening 156 and be retained in the widened region 158 of the lumen 154. Thus, the folded portion 166 of the sleeve 160 may be positioned over the male member 125 of the handle member 120, and the proximal end portion 162 may be secured between the barb 127 of the male member 125 and the cap member 140. The handle member 120 may also have a retaining member formed by an internal shoulder 126 configured to releasably engage the distal end portion 164 when the sleeve 160 is housed in the introducer 100 prior to insertion of the catheter member 200. The handle member 120 may have a distal end portion including a plurality of longitudinal guide members 128 separated by slots 129. A finger grip or pad 130 may be disposed on at least one of the guide members 128 to facilitate manipulation of the introducer 100.

FIG. 10 further illustrates the tubular member 210 of the catheter member 200. In some embodiments, the catheter assembly 10 has the handle member 220 on a distal end portion of the catheter member 200 (as illustrated in FIGS. 1-2). The handle member 220 may have an internal funnel and/or a bag attached to collect a fluid (e.g., urine) flowing through the catheter member 200. In some embodiments, the proximal end portion of the catheter member 200 is open, and the catheter member 200 has a lumen therethrough. The catheter member 200 may be used in urology applications (e.g., intermittent catheters, Foley catheters, endo devices, delivery systems), vascular applications (e.g., sheaths), and other surgical applications. The catheter assembly 10 may be non-lubricated enabled by the non-frictional insertion provided by the everting sleeve 160. The non-lubricated feature prevents spilling and staining of clothing of the user or administer.

As illustrated in FIG. 10, the proximal end portion of the tubular member 210 may include slots or holes 230 extending through a side wall that render the proximal end portion relatively more flexible than the rest of the tubular member 210. The slots 230 may extend at least partially around the perimeter of the tubular member 210. For example, as illustrated in FIG. 10, the slots 230 may extend circumferentially in a pairwise manner, and vary along the proximal end portion of the tubular member 210. Thus, the slots 230 may alternate circumferentially in a pairwise manner along the length of the proximal end portion of the tubular member 210. The density of the slots 230 may, additionally or alternatively, increase proximally along the proximal end portion of the tubular member 210, such that a first length 232 of the tubular member 210 has a greater density of slots 230 than a second length 234 distal of the first length 232. The second length 234 may also have a greater density of the slots 230 than a third length 236 of the tubular member 210 distal of the second length 234. The proximally increasing density of the slots 230 may provide more flexibility at the proximal tip of the tubular member 210 to allow for easier insertion through the urethra, especially in cases where the path of travel is curved or tortuous. In use, the slots 230 would be covered by the sleeve 160 such that the slots 230 do not affect the passage of fluid. The tubular member 210 may further include a tapered and/or rounded proximal tip 240 to engage the sleeve 160. In some embodiments, the sleeve 160 is not secured to (e.g., only releasably engaging) the catheter member 200, such that the sleeve 160 and the catheter member 200 may be inserted and/or removed from the lumen separately.

FIGS. 11A-D illustrate a method of inserting the catheter assembly 10 in a bodily lumen, such as a urethra (U). As illustrated in FIG. 11A, the introducer 100 may act as an introducer aid and facilitate handing of the catheter member 200 and locating/engaging of tissue around the bodily lumen, such as the urethra meatus (UM). The introducer 100 housing the entirety of the sleeve 160 may be place against the urethra meatus (UM). The sleeve 160 may be retained in the introducer 100 between the internal shoulder 126 of the handle member 120 and the closed flaps 142 of the cap member 140. The projection 146 may be initially inserted into the urethra meatus (UM) to align the lumen of the introducer 100 with the urethra (U). The proximal surface 150 and/or side surfaces 151 of the cap member 140 may engage the urethra meatus (UM) preventing further insertion.

As illustrated in FIG. 11B, a proximal end portion of the tubular member 210 may be inserted into the distal opening of the introducer 100. The longitudinal guide members 128 may guide the tubular member 210 through the lumen of the introducer 100. The proximal end portion of the tubular member 210 may engage the distal end portion of the sleeve 160, as further illustrated in FIG. 9. The proximal end portion of the tubular member 210 may lift the distal end portion 164 of the sleeve 160 off the internal shoulder 126 of the introducer 100. The tubular member 210 and the sleeve 160 may exit the proximal opening 147 of the cap member 140 through the flaps 142. As the tubular member 210 translates proximally, the compressed portion 168 gradually unfolds from the proximal to the distal direction and everts over the tubular member 210. The everting of the sleeve 160 prevents transmission of pathogens from the initial location of the urethra meatus (UM) proximally up through the urethra (U) and prevents frictional irritation of the inner epithelial layer. The distal end portion the compressed portion 168 may maintain a compressed configuration on the proximal tip of the tubular member 210 (shown in a cross-section cutaway) producing a soft proximal tip of the catheter assembly 10 to further reduce injury to the sensitive inner epithelial lining of the urethra (U). Even in the compressed configuration, the compressed portion 168 may maintain the lumen 170 such that the compressed portion 168 does not have to be unfolded to allow passage of fluid.

As illustrated in FIG. 11C, the proximal end portion of the catheter member 200 and sleeve 160 may reach the bladder (B). Urine may pass through the lumen of the catheter member 200 through the funnel of the handle member 220 into a toilet or into an optional bag attached to the distal end portion of the catheter member 200. After urine is voided, the catheter member 200 may be removed prior to the sleeve 160. The sleeve 160 may then be removed after the catheter member 200 with reduced comfort due to the pliability of the sleeve 160. However, in some embodiments, the sleeve 160 may be removed with the catheter member 200 in a one-step removal process.

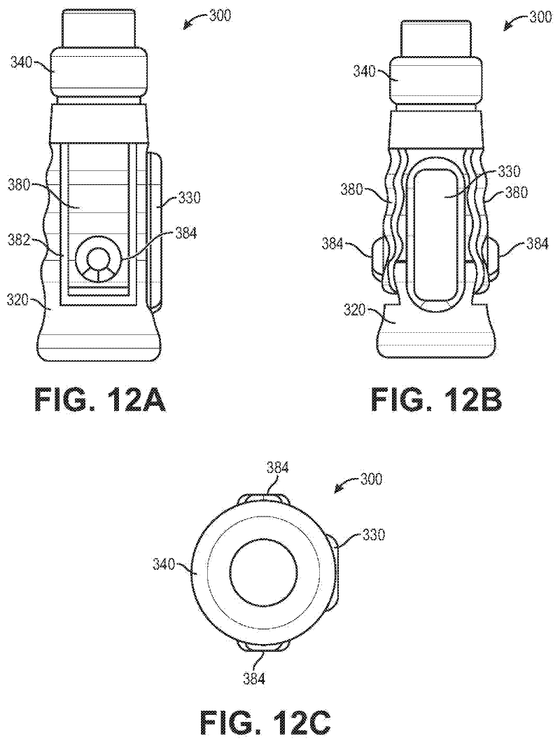

FIGS. 12A-C illustrate views of an introducer 300 of a catheter assembly according to a second embodiment. The introducer 300 includes a handle member 320 and a cap member 340 similar to the introducer 100. The introducer 300 may retain a sleeve in a similar manner (not shown) as discussed above with regard to introducer 100. The above discussion of the handle member 120, 320, the cap member 140, 340, and the sleeve 160 is incorporated herein by reference for sake of brevity. However, the introducer 300 may be configured for one-step removal of the catheter assembly. For example, the handle member 320 may include clamping members 380 on opposing sides of the handle member 320. The clamping members 380 may be configured to be pressed and/or deflected into a lumen of the introducer 300 to clamp the tubular member 210 of the catheter member 200 when inserted into the bodily lumen. Thus, for the catheter member 200 may be removed with the introducer 300 in a one-step process. The clamping members 380 may be attached to the handle member 320 in a cantilever configuration defining a U-shaped slot 382. The clamping members 380 may each include a protrusion 384 to facilitate pressing by the user. The introducer 300 may also include a finger grip or pad 330 on an outer surface of the handle member 320 circumferentially transverse to the clamping members 380. The finger grip or pad 330 may allow undisturbed insertion of the introducer 300 into the opening of the bodily lumen.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A catheter assembly comprising:
a catheter member having a proximal portion and an outer surface;
an introducer having a lumen configured to receive the proximal portion of the catheter member; and
a sleeve having a compressed portion housed in the lumen of the introducer, the sleeve being configured to evert over the outer surface as the catheter member is inserted through the lumen of the introducer.

2. The catheter assembly of claim 1, wherein the proximal portion of the catheter member is configured to push a distal portion of the sleeve proximally, and the compressed portion of the sleeve forms a soft proximal tip of the catheter assembly as the catheter member exits a proximal opening of the introducer.

3. The catheter assembly of claim 1, wherein the sleeve is not secured to the catheter member.

4. The catheter assembly of claim 1, wherein the introducer includes a cap member having an enlarged proximal surface.

5. The catheter assembly of claim 4, wherein the cap member has a retaining member that covers the sleeve when contained in the introducer and deflects as the catheter member is advanced proximally from the introducer.

6. The catheter assembly of claim 5, wherein the retaining member comprises a plurality of flaps.

7. The catheter assembly of claim 5, wherein the retaining member forms a projection extending proximally from the enlarged proximal surface.

8. The catheter assembly of claim 1 wherein the introducer comprises a handle member having a surface configured to be gripped by a user.

9. The catheter assembly of claim 8, wherein the surface of the handle member includes a concave surface and/or one or more ridges.

10. The catheter assembly of claim 8, wherein the proximal portion of the sleeve is secured to the introducer.

11. The catheter assembly of claim 8, wherein the cap member and the handle member are in a mating configuration.

12. The catheter assembly of claim 1, wherein the distal portion of the sleeve is releasably engaged by an internal shoulder of the introducer.

13. The catheter assembly of claim 8, wherein the handle member includes a plurality of elongated guide members extending from a distal portion.

14. The catheter assembly of claim 1, wherein the proximal portion of the catheter member is open, and the catheter member has a lumen therethrough.

15. The catheter assembly of claim 1, wherein the catheter member includes a funnel member on a distal portion.

16. The catheter assembly of claim 1, wherein the catheter assembly is non-lubricated.

17. A method of inserting a catheter assembly of claim 1 into a bodily lumen, the method comprising:
inserting the catheter member into the lumen of the introducer containing the compressed portion of the sleeve; and
everting the sleeve over the catheter member as the catheter member is inserted into the bodily lumen.

18. The method of claim 17, further comprising:
pushing a distal portion of the sleeve proximally; and
forming a soft proximal tip of the catheter assembly with the compressed portion of the sleeve as the catheter member is inserted into the bodily lumen.

19. The method of claim 17, further comprising inserting a projection of the introducer into the bodily lumen.

20. The method of claim 17, further comprising:
retaining the sleeve in the introducer with a retaining member; and
deflecting the retaining member as the sleeve is advanced proximally from the introducer.
